(19) United States
(12) Patent Application Publication
(10) Pub. No.: US 2020/0110083 A1
(43) Pub. Date: Apr. 9, 2020

(54) BARCODED NANOPARTICLES FOR SPECIFIC TARGETING IN VIVO

(71) Applicant: Verily Life Sciences LLC, South San Francisco, CA (US)

(72) Inventors: Mark Audeh, San Francisco, CA (US); Zhan Wang, San Jose, CA (US)

(73) Assignee: Verily Life Sciences LLC, South San Francisco, CA (US)

(21) Appl. No.: 16/593,163

(22) Filed: Oct. 4, 2019

Related U.S. Application Data
(60) Provisional application No. 62/741,921, filed on Oct. 5, 2018.

(51) Int. Cl. G01N 33/543; A61K 9/00; G01N 33/574

(52) U.S. Cl. CPC G01N 33/54346; B82Y 5/00; G01N 33/574; A61K 9/0019

(57) ABSTRACT

Provided herein are compositions and methods for identifying one or more targeted nanoparticles suitable for delivery to one or more organs or tissues of a subject.

### CROSS-REFERENCE TO RELATED APPLICATION

The present application claims the benefit of priority to U.S. Provisional Application No. 62/741,921, filed Oct. 5, 2018, the entire contents of which are incorporated by reference herein for all purposes.

### FIELD

The present disclosure relates to compositions of and methods for the identification of targeted nanoparticles suitable for delivery to one or more organs or tissues of a subject.

### BACKGROUND

The potential for nanoparticle therapeutics has not been fully realized because a targeted nanoparticles specific for a particular cellular antigen were not previously available. As a result, when injected, a very low percentage of current nanoparticle therapeutics make it to the intended organ, tumor, or cell type. Non-specific surface adsorption of biomolecules onto nanoparticles, known as biofouling, and the uptake of nanoparticles by the mononuclear phagocyte system (MPS) and reticuloendothelial system (RES) in vivo lead to substantial reduction in the efficacy and efficiency of target-directed delivery of nanoparticles in biomedical applications. Any molecules targeting these antigens must avoid biofouling and, when targeting a tumor antigen, the particle itself must have the ability to be extravasated from blood vessels into the local tumor microenvironment, while avoiding clearance by RES organs. Further, the performance of a targeting molecule on a nanoparticle is difficult to assess in an in vivo setting, and there are conflicting opinions in the field as to what targeting molecule characteristics might result in a nanoparticle-targeting molecule conjugate that specifically targets a tissue or organ in the subject. Therefore, the field lacks a practical solution for identifying targeted nanoparticles that can be delivered to one or more organs or tissues of a subject.

### SUMMARY

Provided herein is a method of identifying one or more targeted nanoparticles suitable for delivery to one or more organs or tissues of a subject. The method includes the steps of (a) administering to a subject a plurality of targeted nanoparticles, wherein the plurality includes subsets of nanoparticles, wherein each subset is conjugated to a unique non-lipid targeting molecule capable of binding a target antigen in an organ or tissue and wherein each subset comprises a barcode identifier sequence corresponding to the unique non-lipid targeting molecule; (b) obtaining one or more cells from the one or more organs or tissues of the subject; (c) amplifying one or more barcode identifier sequences in the one or more cells obtained in step (b); and (d) identifying the one or more non-lipid targeting molecules associated with the barcode identifier sequence in the one or more cells from the one or more organs or tissues of the subject, thereby identifying one or more targeted nanoparticles suitable for delivery to one or more organs or tissues of the subject. In some embodiments, the method further includes the step of sequencing the amplified barcode identifier sequences from step (c).

In some embodiments, the plurality of targeted nanoparticles comprises between about two and about five hundred nanoparticles. The non-lipid targeting molecule is optionally selected from the group consisting of an antibody, a protein, a peptide, an aptamer or a small molecule. In some embodiments, the antigen is a protein, a lipid or a carbohydrate. Each unique non-lipid targeting molecule optionally binds a different target antigen.

The plurality of nanoparticles optionally comprises two or more nanoparticles, wherein each unique non-lipid targeting molecule conjugated to each of the two or more nanoparticles has a different binding affinity for the same target antigen. In some embodiments, the plurality of nanoparticles comprises two or more subsets of nanoparticles, wherein each subset of nanoparticles has a different size. Each nanoparticle, by way of example, can be between about 50 nanometers and about 500 nanometers in size. In some embodiments, the plurality of nanoparticles comprises two or more subsets of nanoparticles, wherein each subset of nanoparticles has a different chemical composition. Each subset of nanoparticle optionally has a different number of non-lipid targeting molecules conjugated to each subset of nanoparticles.

The barcode identifier sequence optionally further comprises a unique molecular identifier sequence, and the method can further include the step of amplifying one or more unique molecular identifier sequences and/or the step of sequencing the one or more unique molecular identifier sequences. In some embodiments, the method further includes the step of quantifying the one or more barcode identifier sequences or the one or more unique molecular identifier sequences. The barcode identifier sequence is optionally between about five and about one hundred nucleotides in length, and the unique molecular identifier sequence is optionally between about five and about fifty nucleotides in length.

By way of example, the amplification step can be performed by a polymerase chain reaction (PCR). The one or more sequencing steps optionally comprise sequencing by synthesis or high throughput sequencing.

The one or more cells obtained from one or more tissues of the subject can be separated into individual compartments (e.g., wells of a tissue culture plate or) prior to amplification of the barcode identifier sequence(s).

In some embodiments, the one or more cells are lysed prior to amplification of the barcode identifier sequence. The one or more cells can be obtained from one or more tissues or organs selected from the group consisting, for example, of heart, liver, lung, muscle, blood, brain, spleen, skin, pancreas, mouth, esophagus, stomach, small intestine, large intestine, gallbladder and kidney. In some embodiments, the one or more cells obtained from one or more tissues or organs are cancer cells.

### DESCRIPTION OF THE DRAWINGS

The present application includes the following figures. The figures are intended to illustrate certain embodiments and/or features of the compositions and methods and to supplement any description(s) of the compositions and methods. The figures do not limit the scope of the compositions and methods, unless the written description expressly indicates that such is the case.

FIG. 1 shows an exemplary synthesis scheme for making barcoded nanoparticles conjugated to a targeting molecule.

FIG. 2 shows an exemplary scheme for a multiplexed animal model study. In this study, a plurality of targeted nanoparticle(s) are delivered to an animal model and one or more nanoparticles that can target specific organs and/or tumors in the animal model are identified.

FIG. 3 shows photomicrographs (left) showing cell imaging of targeted and barcoded nanoparticles in the presence of A549 cells and a bar graph (right) showing quantification of the cell imaging.

FIG. 4 shows a bar graph of specific binding of EGFR-NP to A549 cells (left column), as measured by qPCR.

FIG. 5 shows a bar graph of blood circulation half-life, in minutes, of nine different lipid formulations with four different barcodes for each lipid formulation.

FIG. 6 shows a bar graph of percent coefficient of variation (% CV) across technical replicates of tissue samples.

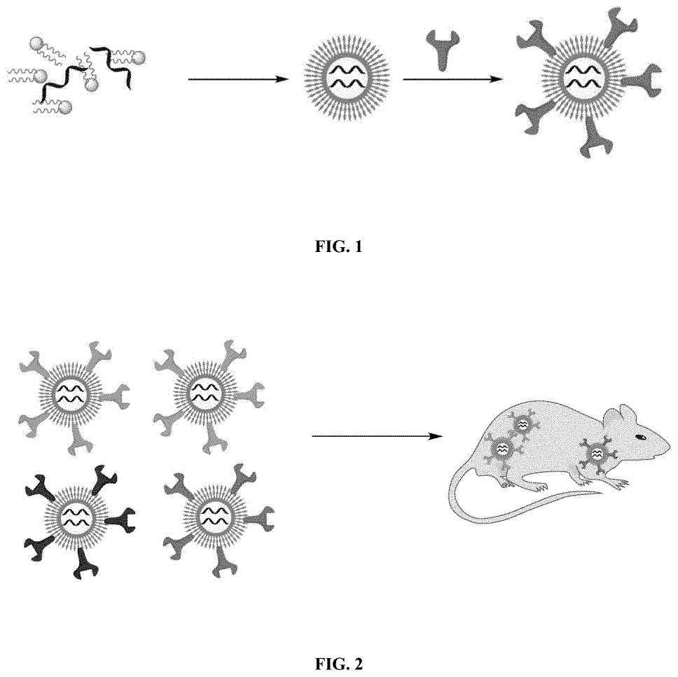

### DETAILED DESCRIPTION

The following description recites various aspects and embodiments of the present compositions and methods. No particular embodiment is intended to define the scope of the compositions and methods. Rather, the embodiments merely provide non-limiting examples that are at least included within the scope of the disclosed compositions and methods.

Provided herein are compositions and methods for the in vivo screening of multiple nanoparticles conjugated to target molecules for the identification of nanoparticle/target molecule combinations that can deliver therapeutic agents to one or more tissues or organs in a subject. Some methods include the steps of (a) administering to a subject a plurality of targeted nanoparticles, wherein the plurality includes subsets of nanoparticles, wherein each subset is conjugated to a unique non-lipid targeting molecule capable of binding a target antigen in an organ or tissue and wherein each subset comprises a barcode identifier sequence corresponding to the unique non-lipid targeting molecule; (b) obtaining one or more cells from the one or more organs or tissues of the subject; (c) amplifying one or more barcode identifier sequences in the one or more cells obtained in step (b); and (d) identifying the one or more non-lipid targeting molecules associated with the barcode identifier sequence in the one or more cells from the one or more organs or tissues of the subject, thereby identifying one or more targeted nanoparticles suitable for delivery to one or more organs or tissues of the subject.

As used herein, the term nanoparticle refers to a particle having a size, i.e., a diameter, of about 1000 nanometers (nm) or less. For example, a nanoparticle can have a size of about 50 nm to about 1000 nm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, or about 50 nm to about 100 nm. Nanoparticles include nanoparticles comprising a compartment for encapsulation of an agent, for example, a barcode identifier sequence, nanoparticles comprising a conjugated targeting molecule attached to the outside of the nanoparticle and nanoparticles comprising an incorporated barcode identifier sequence. An incorporated barcode identifier sequence can be completely or partially located in the interior space of the nanoparticle. Incorporation is also referred to herein as encapsulation wherein the barcode identifier sequence is entirely contained within the interior space of the nanoparticle.

As used throughout, nanoparticles can be, but are not limited to, lipid nanoparticles, for example, liposomes or non-liposomal lipid nanoparticles (for example, lipid nanoparticles with a non-aqueous core (LNPs)), dendrimers, polymeric micelles, nanocapsules or nanospheres, to name a few.

The lipid nanoparticles of the present invention, for example, liposomes, can be made by any suitable method known to or later discovered by one of skill in the art. As used herein, the term liposome refers to an aqueous or aqueous-buffered compartment enclosed by a lipid bilayer. In general, liposomes can be prepared by a thin film hydration technique followed by a few freeze-thaw cycles. Liposomal suspensions can also be prepared according to methods known to those skilled in the art. Exemplary methods for the preparation of liposomes are described in Akbarzadeh et al. (“Liposome: classification, preparation and applications,” Nanoscale Res. Lett. 8(1): 102 (2013)) which is hereby incorporated by reference in its entirety.

In general, a variety of lipid components can be used to make the lipid nanoparticles, for example, liposomes or LNPs, of the present invention. These include neutral lipids that exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. Synthetic derivatives of any of the lipids described herein can also be used to make lipid nanoparticles. Lipid nanoparticles can also comprise a sterol, for example, cholesterol. Lipid nanoparticles can also comprise a cationic lipid which carries a net positive charge at about physiological pH. Such cationic lipids include, but are not limited to, N,N-dioleyl-N,N-dimethylammonium chloride (DODAC); N-(2,3-dioleyloxy)propyl-N,N—N-triethylammonium chloride (DOTMA); N,N-distearyl-N,N-dimethylammonium bromide (DDAB); N-(2,3-dioleoyloxy)propyl)-N,N,N-trimethylammonium chloride (DOTAP); 1,2-Dioleyloxy-3-trimethylaminopropane chloride salt (DOTAP.Cl); 3.beta.-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (DOGS), 1,2-dileoyl-sn-3-phosphoethanolamine (DOPE), 1,2-dioleoyl-3-dimethylammonium propane (DODAP), N,N-dimethyl-2,3-dioleyloxy)propylamine (DODMA), and N-(1,2-dimyristyloxyprop-3-yl)-N,N-dimethyl-N-hydroxyethyl ammonium bromide (DMRIE). Anionic lipids are also suitable for use in lipid nanoparticles described herein. These include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

In some embodiments, the lipid nanoparticle, for example, a liposome or LNP comprises phosphatidylcholine, phosphatidylethanolamine, phosphatidylserine, phosphatidylinositol, phosphatidic acid, phosphatidylglycerol, palmitoyloleoyl phosphatidylcholine, lysophosphatidylcholine, lysophosphatidylethanolamine, dimyristoylphosphatidylcholine (DMPC), dipalmitoylphosphatidylcholine (DPPC), dioleoylphosphatidylcholine, di stearoylphosphatidylcholine (DSPC), dilinoleoylphosphatidylcholine, a 1,2-distearoyl-sn-glycero-3-phosphoethanolamine (DSPE) conjugated polyethylene glycol (DSPE-PEG), a sphingomyelin, cholesterol, or any combination thereof. In some embodiments, PEG can be PEG-molecular weight (MW)500 to PEG-MW20000. In some embodiments, the lipid nanoparticle comprises DSPC, DSPE-PEG(MW2000) and cholesterol. In some embodiment the ratios of phospholipid 1:phospholipid 2:sterol:PEG-lipid can be about 30:30:35:5, but can vary. For example, phospholipid 1 and phospholipid 2 are optionally the same or different. Where phospholipid 1 and 2 are the same, the ratio can be expressed, for example, as either 30:30:35:5 or as 60:35:5. The amount of phospholipid 1 or phospholipid 2 can be, by way of example, from about 5% to about 60%. For example, the amount of phospholipid 1 or phospholipid 2 can be about 5%, 10%, 15%, 20%, 25%, 30%, 35%, 40%, 45%, 50%, 55%, 60% or any percentage in between these percentages. The amount of a sterol can be from about 25% to about 40%, for example, about 25%, 30%, 35%, 40%, or any percentage in between these percentages. The amount of PEG-conjugated lipid can be about 5% to about 10%, for example about 5%, 6%, 7%, 8%, 9%, 10% or any percentage in between these percentages. In some examples, a lipid ratio of 55:5:35:5 can also be used. In some embodiments, the lipid nanoparticle does not include cholesterol and/or PEG-lipid.

Optionally, the chemical composition of any of the nanoparticles described herein is selected or designed to enhance the stability of the contents of a nanoparticle, for example, a barcode identifier sequence contained therein. Depending on its chemical composition, the nanoparticle can allow the barcode identifier sequence to reach the target cell and/or preferentially allow the barcode identifier sequence to reach the target cell, or alternatively limit the delivery of the barcode identifier sequence to other undesired target sites or cells. One of skill in the art would know how to select and/or prepare a nanoparticle with the desired properties (e.g., size, charge, chemical composition and/or pH) to effectively target one or more cells in one or more tissues of a subject. For example, if the target cell is a hepatocyte, the nanoparticle can be optimized to effectively deliver the nanoparticle to a hepatocyte. In another example, if the target cell is a neural cell, one of skill one of skill in the art would consider penetration of the blood brain barrier and/or direct delivery of the nanoparticle when selecting and/or preparing a nanoparticle. In another example, when targeting tumor cells, one of skill in the art would select and/or prepare nanoparticles that target tumor cells while reducing toxic effects on surrounding normal tissue.

As used throughout, the term plurality of nanoparticles means two or more nanoparticles. In some embodiments, the plurality of targeted nanoparticles comprises between about two and about five hundred nanoparticles. For example, the plurality of targeted nanoparticles can comprise between two and ten, between two and twenty, between two and thirty, between two and forty, between two and fifty, between two and sixty, between two and seventy, between two and eighty, between two and ninety, or between two and one hundred, between two and one hundred ten, between two and one hundred twenty, between two and one hundred thirty, between two and one hundred forty, between two and one hundred fifty, between two and one hundred sixty, between two and one hundred seventy, between two and one hundred eighty, between two and one hundred ninety, between two and two hundred targeted nanoparticles, between two and two hundred fifty nanoparticles, between two and three hundred targeted nanoparticles, between two and three hundred fifty targeted nanoparticles, between two and four hundred targeted nanoparticles, between two and four hundred fifty targeted nanoparticles or between two and five hundred targeted nanoparticles.

By way of example, and without meaning to be limited, the plurality of targeted nanoparticles can comprises about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 nanoparticles.

As used herein, the term subset refers to one or more nanoparticles in the plurality of targeted nanoparticles that are conjugated to a unique or different non-lipid targeting molecule as compared to other nanoparticles in the plurality. A subset of nanoparticles can comprise between one and about 250 nanoparticles or more. In some embodiments, a subset comprises about 5, 10, 20, 30, 40, 50, 60, 70, 80, 90, 100 nanoparticles or more.

As used throughout, a non-lipid targeting molecule is a molecule that has a binding affinity for an antigen, optionally a specific binding affinity, and can include, but is not limited to, an antibody, an aptamer, a protein, a peptide or a small molecule. As used throughout, an antigen can be, but is not limited to a protein, a lipid or carbohydrate. Optionally, the antigen can be a cell surface antigen. In the methods provided herein, each of the nanoparticles in a subset is conjugated to the same unique non-lipid targeting molecule. For example, each of the nanoparticles in a subset can be conjugated to a non-lipid targeting molecule, wherein each non-lipid targeting molecule binds the same protein, lipid or carbohydrate of a cell.

As used throughout, the term antibody encompasses, but is not limited to, a nanobody, a whole immunoglobulin (i.e., an intact antibody) of any class, including polyclonal and monoclonal antibodies, as well as fragments of antibodies that retain the ability to bind their specific antigens. Also useful are conjugates of antibody fragments and antigen-binding proteins (single chain antibodies) as described, for example, in U.S. Pat. No. 4,704,692, the contents of which are hereby incorporated by reference in their entirety.

As used throughout, an aptamer is an oligonucleotide (single stranded DNA or single stranded RNA) or a peptide molecule that selectively bind to a target antigen. See, for example, Lakhin et al. “Aptamers: Problems, Solutions and Prospects,” Acta Naturae 5(4): 34-43 (2013); and Reverdatto et al., “Peptide aptamers: development and applications,” Curr. Top Med. Chem. 15(12): 1082-101 (2015))) hereby incorporated in their entireties by this reference.

As used herein, the terms specifically binds or selectively binds mean binding that is measurably different from a non-specific or non-selective interaction. Specific binding can be measured, for example, by determining binding of a molecule to a target antigen compared to binding of a control molecule. Specific binding can be determined by competition with a control molecule that is similar to the target antigen, such as an excess of non-labeled target antigen. In that case, specific binding is indicated if the binding of the labeled target to a probe is competitively inhibited by the excess unlabeled target antigen.

A unique non-lipid targeting molecule can be a non-lipid targeting molecule with a unique structure (e.g., chemical structure, amino acid sequence or nucleic acid sequence), a unique binding affinity for a target antigen or both. Optionally, from two to about five hundred unique non-lipid targeting molecules can be conjugated to a nanoparticle. For example, from about five to about ten, about five to about twenty, about five to about thirty, about five to about forty, about five to about fifty, about five to about sixty, about five to about seventy, about five to about eighty, about five to about ninety, about five to about one hundred, about five to about one hundred fifty, about five to about two hundred, about five to about two hundred fifty, about five to about three hundred, about five to about three hundred fifty, about five to about four hundred, about five to about four hundred fifty, or about five to about five hundred unique non-lipid targeting molecules can be conjugated to one or more nanoparticles described herein. For example, about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, or any number of non-lipid targeting molecules in between these numbers can be conjugated to one or more nanoparticles described herein.

Non-lipid targeting molecules can be conjugated to nanoparticles by a number of methods known in the art (e.g., Arruebo et al. “Antibody-Conjugated Nanoparticles for Biomedical Applications,” Journal of Nanomaterials vol. 2009, Article ID 439389 (2009)). Nanoparticles can also be conjugated to non-lipid targeting molecules via a streptavidin/biotin bond, thiol/maleimide chemistry, azide/alkyne chemistry, tetrazine/cyclooctyne chemistry, and other click chemistries. These chemical handles are prepared either during phosphoramidite synthesis or post-synthesis. As used herein, the term click chemistry refers to biocompatible reactions intended primarily to join substrates of choice with specific biomolecules. Click chemistry reactions are not disturbed by water, generate minimal and non-toxic byproducts, and are characterized by a high thermodynamic driving force that drives it quickly and irreversibly to high yield of a single reaction product, with high reaction specificity. Methods for chemical conjugation of targeting molecules are described in the Examples.

In some embodiments, the plurality of nanoparticles comprises two or more subsets of nanoparticles, wherein each subset is conjugated to a lipid targeting molecule that binds to or is capable of binding a different or unique target antigen. For example, the plurality of nanoparticles can comprise a first subset of nanoparticles, wherein each nanoparticle in the subset is conjugated to a lipid targeting molecule is capable of binding or binds to a first antigen, and a second subset of nanoparticles, wherein each nanoparticle in the subset is conjugated to a lipid targeting molecule that is capable of binding or binds to a second target antigen. The plurality of nanoparticles optionally comprises two or more subsets of nanoparticles, wherein each subset is conjugated to a lipid targeting molecule that binds the same target antigen, and wherein each subset has a different binding affinity for the same target antigen. For example, the plurality of nanoparticles can comprise a first subset of nanoparticles, wherein each nanoparticle in the subset is conjugated to a lipid targeting molecule that has a first binding affinity for a target antigen, and a second subset of nanoparticles, wherein each nanoparticle in the subset is conjugated to a lipid targeting molecule that has a second binding affinity to the target antigen. In other embodiments, the plurality of nanoparticles comprises four or more subsets of nanoparticles, wherein two or more subsets of nanoparticles are conjugated to a lipid targeting molecule that binds to a first target antigen, and wherein the lipid targeting molecule conjugated to each of the two or more subsets of nanoparticles has a different binding affinity for the first target antigen; and wherein two or more subsets of nanoparticles are conjugated to a lipid targeting molecule that binds to a second target antigen, wherein the lipid targeting molecule conjugated to each of the two or more subsets of nanoparticles has a different binding affinity for the second target antigen.

The plurality of nanoparticles optionally comprises two or more subsets of nanoparticles, wherein each subset of nanoparticles has a different size. Given variability in size of nanoparticles within a set or subset, references to size or diameter generally refers herein to the average size or diameter. A different size of particles in one subset as compared to other subsets in the plurality refers to a difference in the average size so as to be distinguishable from larger or smaller subsets of nanoparticles in the plurality. Thus, such a difference will necessarily be larger or smaller than the range of variation within the subsets.

In some embodiments, the plurality of nanoparticles comprises two or more subsets of nanoparticles, wherein each subset of nanoparticles has a different chemical composition. Again, such difference in composition should be distinguishable and beyond the range or variation within the subsets. In some embodiments, the plurality of nanoparticles comprises two or more subsets of nanoparticles, wherein each subset of nanoparticles is conjugated to a different number of unique non-lipid targeting molecules.

In the methods provided herein, each nanoparticle in the plurality of nanoparticles or in a subset of the plurality comprises a barcode identifier sequence corresponding to the unique non-lipid targeting molecule conjugated to the nanoparticle. In some embodiments, the unique barcode identifier sequence is encapsulated in the nanoparticle. In the methods described herein, incorporation of unique barcode identifier sequences into the nanoparticles allows the identification of non-lipid targeting molecules that can be used to target nanoparticles to a tissue or organ of a subject, as well as one or more nanoparticles that are suitable for delivery to one or more tissues or organs of a subject. Assays for identifying barcode identifier sequences include, but not limited to, microarray systems, PCR, nucleic acid hybridization, or high throughput sequencing.

The term barcode identifier sequence refers to a sequence that corresponds to the unique non-lipid targeting molecule that binds to a target antigen. After administration of the plurality of targeted nanoparticles to the subject, amplification and/or sequencing methods can be used to identify barcode sequences and the unique non-lipid targeting molecules associated with the barcode sequences. Since each unique non-lipid targeting molecule is conjugated to a nanoparticle, identification of the unique non-lipid targeting molecule allows identification of a nanoparticles suitable for delivery to one or more organs or tissues of the subject. The barcode identifier sequence may be between about five to about one hundred nucleotides in length. For example, the antigen-binding molecule identifier sequence may be between about 5 and about 10, between about 10 and about 20, between about 20 and about 30, between about 30 and about 40, between about 40 and about 50 nucleotides in length, between about 50 and about 60 nucleotides in length, between about 60 and about 70 nucleotides in length, between about 70 and about 80 nucleotides in length, between about 80 and about 90 nucleotides in length or between about 90 and about 100 nucleotides in length.

In any of the methods provided herein, one or more nanoparticles described herein can comprise between one and about 5000 barcode identifier sequence molecules. For example, one or more nanoparticles can comprise about 5, 10, 20, 30, 40, 50, 60, 70, 80, 90, 100, 200, 300, 400, 500, 1000, 1500, 2000, 2500, 3000, 3500, 4000, 4500, 5000 or any number of barcode identifier sequence molecules in between these numbers.

As used throughout, the term barcode identifier sequence can be used interchangeably with the term barcode or barcode sequence, and the terms have the same meaning. In any of the methods provided herein, the barcode can comprise deoxyribonucleic acids (DNA) or ribonucleic acids (RNA) or both, in either single- or double-stranded form. Optionally, the barcode can comprise synthetic, non-natural or altered nucleotide bases. Optionally, the barcode can further comprise a nucleic acid sequence corresponding to the target antigen bound by the unique non-lipid targeting molecule. Optionally, the barcode can also include a nucleic acid sequence corresponding to the chemical composition, the size of the nanoparticle, and/or the number of unique non-lipid targeting molecules conjugated to the nanoparticle. Optionally, the barcode can also include one or more nucleic acid sequences for amplification and/or sequencing, for example, PCR handles.

Optionally, the barcode further comprises a unique molecular identifier sequence. The unique molecular identifier sequence can be between about five to about fifty nucleotides in length. For example, the molecular identifier sequence can be between about 5 and about 10, between about 10 and about 20, between about 20 and about 30, between about 30 and about 40, or between about 40 and about 50 nucleotides in length or any number of nucleotides within these ranges. The term unique molecular identifier sequence refers to a sequence that can be used to identify a specific oligonucleotide through amplification and/or sequencing methods. The use of unique molecular identifier sequences (UMIs) for amplification and high throughput sequencing reduces bias in quantification of the sequences after amplification. Due to the high sequence diversity of UMIs, no two reads in the library should contain the same UMI, unless they are duplicated in the PCR process. Such duplicates are collapsed into one read so that an undistorted representation of the original pre-PCR library is obtained. Since the UMIs are part of the barcode, they can be automatically incorporated into a sequencing library without additional tagging.

Optionally, the barcode is labeled with a detectable label, for example, with biotin, a radiolabel, or a fluorescent label. Optionally. the barcode sequence does not include nucleotide sequences of 10 or more bases that can associate with a naturally occurring nucleotide sequence in the one or more cells being targeted using the methods and compositions described herein. Optionally, the barcode comprises a sequence that is not substantially identical or complementary to cellular genomic DNA to prevent hybridization of the barcode with the cell's genomic DNA and/or to prevent false positive amplification results.

The methods and compositions provided herein can be used for in vivo targeting and delivery of the contents of a nanoparticle(s), for example, barcodes, to one or more cells of a subject. These cells include but are not limited to, hepatocytes, epithelial cells, hematopoietic cells, endothelial cells, lung cells, ductal cells, bone cells, stem cells, mesenchymal cells, neural cells, cardiac cells, adipocytes, vascular smooth muscle cells, myoepithelial cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells. As used herein, tumor cells include primary cancer cells as well as metastatic cells.

In the methods provided herein, a cell-containing sample, i.e., a sample containing one or more cells, are obtained from a subject. The subject can be a mammalian subject. Exemplary subjects include, but are not limited to humans, monkeys, dogs, cats, mice, rats, cows, horses, camels, goats and sheep. In some embodiments, the subject is a human. In some embodiments, the subject has or is suspected of having a tumor, e.g., a malignant tumor.

The one or more cells can be obtained from any part of the subject, for example, from one or more organs or tissues of the subject. For example, and not to be limiting, the one or more cells can be from one or more tissues or organs selected from the group consisting of heart, liver, lung, muscle, testes, blood, brain, spleen, breast, skin, pancreas, mouth, esophagus, stomach, small intestine, large intestine, gallbladder and kidney of the subject. The one or more cells can be obtained from cancerous tissue, e.g., a tumor, or from non-cancerous tissue. The one or more cells can be obtained from one or more cells from one or more organs or tissues of a healthy subject or a subject with a particular disease or disorder. In some embodiments, the one or more cells can be obtained from one or more organs or tissues of a subject with cancer. Optionally, the one or more cells can be obtained from a biopsy of a tissue or organ of the subject.

In some embodiments, a population of cells, i.e., two or more cells, is obtained from a tissue or organ of the subject. Optionally, one or more populations of cells can be obtained from one or more tissues or organs of the subject. Optionally, a subpopulation of one or more cells can be isolated from a population of cells obtained from a tissue or organ of the subject, for example, by fluorescence activated cell sorting (FACS), magnetic-activated cell sorting (MACS), ELISA or microfluidic based sorting to isolate one or more specific cell types from the population of cells. By isolating specific cell types from a population of cells, the methods provided herein can be used to identify nanoparticles suitable for delivery to one or more specific cell types, or subpopulations of cells, in a tissue or organ of the subject. For example, tumor cells can be isolated using one or more cancer markers from a sample containing a population of liver cells comprising cancerous and non-cancerous cells. Following isolation, the barcode identifier sequences can be identified. If a barcode identifier sequence found in the tumor specific cells is not found to any appreciable (or detectable) extent in noncancerous cells from the population, the nanoparticle comprising the non-lipid targeting molecule associated with the barcode identifier sequence is suitable for specifically delivering a therapeutic agent to tumor cells in the liver of a subject with liver cancer. This example is not limited to liver cells, as similar methods can be used to identify nanoparticles suitable for delivery to specific cell types in any tissue or organ of a subjects.

In some embodiments, the methods include allowing for a sustained amount of time for the plurality of nanoparticles to bind one or more target antigens on one or more cells in the subject and to deliver the barcode identifier sequence to the one or more cells, prior to obtaining one or more cells from the subject. Thus, the nanoparticles are delivered in vivo to the one or more tissues or organs of the subject and, after a time sufficient for nanoparticle binding has passed, samples containing cells are obtained from the subject. The sustained amounts of time before obtaining the one or more cells from the subject will depend on the subject, the nature of any disorder or condition present in the subject, the type of barcode identifier sequence and/or the amount of barcode identifier sequence used, the type of nanoparticles, tissue or cells to be targeted, and the like and can be determined by standard clinical techniques known to a person skilled in the art. For example, and not to be limiting, one or more cells can be obtained from the subject about 12, 24, 30, 48 hours, or longer after administration of the plurality of nanoparticles to the subject. In some embodiments, one or more cells are obtained from the subject between about 24 and about 72 hours after administration of the plurality of nanoparticles. In some embodiments, one or more cells are obtained from the subject between about 24 and about 48 hours after administration of the plurality of nanoparticles.

In some embodiments, one or more non-lipid targeting molecules are identified by amplifying the one or more barcode identifier sequences in one or more cells from a tissue or organ of the subject. Optionally, the unique molecular identifier sequence is amplified. One of skill in the art would know how to design amplification primers that specifically amplify the barcode identifier sequence and/or the unique molecular identifier sequence. Optionally, the amplification primers are designed so that detection of an amplified barcode identifier sequence in a cell is sufficient to identify the barcode identifier sequence, thereby identifying the non-lipid targeting molecule and the nanoparticle that delivered the barcode identifier sequence to the cell. In certain embodiments, the amplification is performed using polymerase chain reaction (PCR), for example, quantitative PCR.

Optionally, the one or more amplified barcode sequences and/or unique molecular identifier sequences are quantified. By determining the number of one or more barcode identifier sequences in a cell from a tissue or organ of the subject, one of skill in the art can determine whether a nanoparticle can deliver its contents to a specific tissue or organ and/or assess the efficacy of delivery of a nanoparticle to specific tissues or organs of the subject. For example, a plurality of nanoparticles conjugated to a unique non-lipid targeting molecule that targets a specific cell surface antigen can be administered to the subject. After administration, the barcode identifier sequence corresponding to the non-lipid targeting molecule can be amplified in a sample containing one or more cells from the liver, kidney and brain of the subject. Barcodes that accumulate in a cell(s) are indicative of nanoparticles that can be used to target the cell(s) for treatment of a disease or disorder that affects the tissue or organ comprising the cell(s). If, for example, there are 100 barcode identifier sequences in the one or more cells from the liver, 50 barcode identifier sequences in the one or more cells from the kidney, 10 barcode identifier sequences in the one or more cells from the brain, and zero barcode identifier sequences in the pancreas, one of skill in the art would determine that, although the contents of the nanoparticle can be delivered to and accumulate in the liver, kidney and brain of the subject by targeting the specific cell surface antigen, the barcode identifier sequences preferentially accumulate in the liver as compared to accumulation in the kidney or brain of the subject. One of skill in the art would also determine that the nanoparticle is not effective for administration to the pancreas. Depending on the information associated with the barcode identifier sequence encapsulated by a nanoparticle, for example, information regarding size, chemical composition and/or the number of non-lipid targeting molecules conjugated to the nanoparticle, one of skill in the art can also determine the number of barcodes associated with a nanoparticle having a particular chemical composition, size and/or number of non-lipid targeting molecules conjugated to the nanoparticle via amplification and/or sequencing of the barcode identifier sequence. Barcode identifier sequences comprising a nucleic acid sequence associated with a specific chemical composition, size and/or number of non-lipid targeting molecules conjugated to the nanoparticle that accumulate, preferentially or non-preferentially, in one or more cells of one or more tissues or organs of the subject allow identification of nanoparticle with properties suitable for delivery to the one or more tissues or organs of the subject.

In some embodiments, information from a barcode(s) can be used to extrapolate the antibody-particle PK, antibody-particle distribution, antibody-particle localization and/or antibody-particle penetration into organs/tumor. For example, in an exemplary animal study, nanoparticles are administered to rodents via tail vein injection. Blood draws are collected at different time points during the experiment. After 4-24 hours, the animals are euthanized and blood is removed via cardiac puncture. Individual organs such as lung, heart, brain, bone marrow, spleen, kidney, etc. are harvested and either processed immediately or frozen immediately. Each organ is individually homogenized and processed through an extraction column designed to isolate smaller DNA (Clarity OTX). The eluent is harvested and qPCR is performed to determine barcode content. Indexing sequences are added to each sample to uniquely identify its tissue of origin, animal/sample number, time point, etc. Samples are pooled and sequenced on Illumina MiSeq or HiSeq (San Diego, Calif.). Percent injected dose in a given organ or blood sample can be estimated based on sequencing nanoparticle dose and comparing this dose to sequencing of blood or organ aliquots.

As used throughout, by preferentially accumulates means that more barcode identifier sequences accumulate in one cell type as compared to another cell type. For example, such preferential accumulation may be across organs or tissues (e.g., liver instead of pancreas), across pathologic/normal cells (e.g., cancer versus non-cancerous), or across cells within the same tissue or organ (e.g., lymphocytes as opposed to monocytes). By identifying barcodes that preferentially accumulate, one of skill in the art can identify targeted nanoparticles that, when administered to the subject, preferentially deliver their encapsulated contents, for example, a therapeutic agent, to a particular cell type or a particular tissue or organ of the subject. In some embodiments, the barcode identifier sequences preferentially accumulate in a tumor cell(s) of a tissue or organ in the subject. Depending on the tissue or organ, once barcodes are identified in the tumor(s) cell, one of skill in the art can select or prepare targeted nanoparticles that comprise one or more therapeutic agents for delivery as nanoparticle payload to a tumor cell(s) in the tissue or organ of the subject. Depending on the type of tumor, i.e., the type of cancer, the one or more therapeutic agents can comprise one or more selected chemotherapeutic agents.

As used herein, a tumor cell is sometimes used interchangeably with cancer cell but also encompasses non-malignant (non-cancerous) cells exhibiting increased proliferation as compared to a normal cell. By way of example, the tumor cell is selected from the group consisting of a hematological cancer, a lymphoma, a myeloma, a leukemia, a neurological cancer, melanoma, breast cancer, a prostate cancer, a colorectal cancer, lung cancer, head and neck cancer, a gastrointestinal cancer, liver cancer, pancreatic cancer, a genitourinary cancer, a bone cancer, renal cancer, and a vascular cancer. In some embodiments, the tumor cell is selected from the group consisting of Kaposi's sarcoma, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

Examples of chemotherapeutic agents include, but are not limited to, antineoplastic agents such as Acivicin; Aclarubicin; Acodazole Hydrochloride; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; 5-Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin C; Mitosper; Mitotane; Mitoxantrone; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Other chemotherapeutic agents that can be used include, sorafenib, brivanib, sunitinib, linifanib, erlotinib, everolimus, ramucirumab, regorafenib, lenvatinib, cabozantinib, tivantinib, apatinib, to name a few.

Optionally, cells in a cell-containing sample can be lysed prior to amplification to produce a cell lysate that includes the contents of the lysed cells, for example, proteins, nucleic acids, and fragments thereof. Some of the methods provided herein can comprise lysing a population of cells or individual cells obtained from one or more tissues or organs of the subject with an agent that extracts nucleic acids from the cells. The nucleic acids can comprise DNA and/or RNA.

Methods for lysing cells are known in the art and include, but are not limited to, mechanical disruption of cell membranes, for example, by repeated thawing and freezing, sonication, bead homogenization, pressure, or filtration. Cells can also be lysed with a solution containing a detergent, for example, including, but not limited to Triton X-100, Triton-X114, NP-40, Brij-35, Brij-58, Tween 20, Tween 80, Octyl glucoside, Octyl thioglucoside, sodium dodecyl sulfate (SDS), CHAPS, and CHAPSO, to name a few. Cells can also be lysed by heating the cells to about 70-90° C. In some embodiments, the cell-containing sample is treated with Proteinase K prior to or concurrently with the lysing of the cell or cells in the sample.

In some embodiments, one or more cells from a tissue or organ of the subject are separated into individual compartments prior to lysing the single cell in each compartment. In some embodiments, the compartment may be a well of a tissue culture plate or a microfluidic droplet.

The barcode sequences and/or the unique molecular identifier sequences are optionally sequenced. Sequencing methods include, but are not limited to, shotgun sequencing, bridge PCR, Sanger sequencing (including microfluidic Sanger sequencing), pyrosequencing, massively parallel signature sequencing, nanopore DNA sequencing, single molecule real-time sequencing (SMRT) (Pacific Biosciences, Menlo Park, Calif.), ion semiconductor sequencing, ligation sequencing, sequencing by synthesis (Illumina, San Diego, Ca), Polony sequencing, 454 sequencing, solid phase sequencing, DNA nanoball sequencing, heliscope single molecule sequencing, mass spectroscopy sequencing, pyrosequencing, Supported Oligo Ligation Detection (SOLiD) sequencing, DNA microarray sequencing, RNAP sequencing, tunneling currents DNA sequencing, and any other DNA sequencing method identified in the future. One or more of the sequencing methods described herein can be used in high throughput sequencing methods. As used herein, the term high throughput sequencing refers to all methods related to sequencing nucleic acids where more than one nucleic acid sequence is sequenced at a given time.

In some embodiments, the amplification products from one or more cells from each tissue or organ are pooled prior to sequencing. For example, one or more cells obtained from the liver of a subject can be pooled prior to sequencing the amplification products obtained from the one or more liver cells of the subject; one or more cells obtained from the kidney of a subject are pooled prior to sequencing the amplification products obtained from the one or more kidney cells of the subject; and one or more cells obtained from the brain of a subject are pooled prior to sequencing the amplification products obtained from the one or more brain cells of the subject, etc.

In embodiments where the barcode identifier sequence includes a nucleic acid sequence corresponding to the chemical composition, the size of the nanoparticle, and/or the number of unique non-lipid targeting molecules conjugated to the nanoparticle, sequencing of the barcode identifier sequence allows identification of the lipid-targeting molecule associated with the barcode identifier sequence, the chemical composition of the nanoparticle comprising the barcode identifier sequence, the size of the nanoparticle comprising the barcode identifier sequence and/or the number of non-lipid targeting molecules conjugated to the nanoparticle comprising the barcode identifier sequence.

### Compositions

Compositions comprising one or nanoparticles, including a plurality of nanoparticles of the present disclosure, and a pharmaceutically acceptable carrier are also provided. The compositions may further comprise a diluent, solubilizer, emulsifier, preservative, and/or adjuvant to be used with the methods disclosed herein. Once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In certain embodiments, the formulation components are present in concentrations that are acceptable for administration. In certain embodiments, buffers are used to maintain the composition at physiological pH or at a slightly lower pH, typically within a pH range of from about 5 to about 8.

Solid dosage forms for oral administration include capsules, tablets, pills, powders, and granules. In such solid dosage forms, the nanoparticles are mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

The nanoparticles may be formulated in dosage unit form for ease of administration and uniformity of dosage. For any nanoparticle disclosed herein, the dose or amount of nanoparticles administered to the subject can be estimated in cell culture assays or in one or more animal models, for example, mice, rabbits, dogs, or pigs. The animal model is also used to achieve a desirable concentration range and route of administration. Such information can then be used to determine useful doses and routes for administration in subjects.

### Administration

The route of administration of the nanoparticles disclosed herein can be in accord with known methods, e.g. orally, through injection by intravenous, intraperitoneal, intracerebral (intra-parenchymal), intracerebral, intratumoral, intraventricular, intramuscular, subcutaneously, intra-ocular, intraarterial, intraportal, intranasal, intralesional routes; by sustained release systems or by implantation devices. In certain embodiments, the compositions can be administered by bolus injection or continuously by infusion, or by implantation device.

The administration amount of the nanoparticles may have various ranges thereof depending on weight, age, gender, health condition, diet, administration time, method, excretion rate, the severity of disease, and the like, of a subject, and may be easily determined by a general expert in the art.

As used herein, intranasal administration means delivery of the compositions into the nose and nasal passages through one or both of the nostrils and can comprise delivery by a spraying mechanism or droplet mechanism, or through aerosolization of the compositions. In some embodiments, solutions can be nebulized. Administration of the compositions by inhalant can be through the nose or mouth via delivery by a spraying or droplet mechanism. Delivery can also be directly to any area of the respiratory system (e.g., lungs) via intubation.

The composition can be administered locally, e.g., during surgery or topically. Optionally local administration is via implantation of a membrane, sponge, or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration. The number of nanoparticles administered to a subject can be calculated, for example, by using a nanoparticle tracking instrument and analysis. For example, nanoparticles can be counted and sized using Nanosight (Malvern Instruments, Malvern, UK).

Disclosed are materials, compositions, and ingredients that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### EXAMPLES

### Nanoparticles

Liposomes were prepared using a standard thin film evaporation method. 1,2-dipalmitoyl-sn-glycero-3-phosphoethanolamine (DPPE, Avanti Polar Lipids, Alabaster, Ala.), cholesterol (Sigma Aldrich, St. Louis, Mo.), and methoxy-PEG-DSPE (DSPE-mPEG, Nanocs, Boston, Mass.), and DPPE-Cy5.5 were dissolved in a 2:1 chloroform/methanol mix at a molar ratio of 60:35:4.9:0.1. Click lipids can also be added to the organic solution to facilitate downstream covalent conjugation. The solution was heated to 37° C. to ensure complete dissolution. The solvent was then removed via rotary evaporation until a thin film was formed. The film was desiccated overnight under vacuum prior to rehydration in phosphate buffered saline (PBS). The rehydrated lipid film was sonicated at 69° C. for one hour. While still hot, the lipid solution was extruded through three successive syringe filters (0.45 um, 0.2 um, and finally 0.1 μm) to obtain 100 nm liposomes. Liposomes were washed by centrifugal filtration or tangential flow filtration (TFF) with a 100 kDa MWCO membrane to remove unincorporated lipids.

### Barcodes

Barcoded liposomes were synthesized using the protocol described above. After a thin film was made, a PBS solution containing DNA, RNA, or modified nucleic acid barcodes was added to the film and used as the rehydration buffer. In alternative methods based on precipitation, a nucleic acid barcode can be dissolved in the aqueous phase, while the lipids are dissolved in the organic phase. Unincorporated DNA barcodes were removed using dialysis, tangential flow filtration (TFF) or centrifugation using MWCO filters.

DNA barcode molecules were ordered from a commercial vendor. Barcodes included a unique barcode sequence flanked by conserved amplification primers. A unique molecular identifier sequence (UMI) was added, adjacent to the unique barcode, to account for amplification bias. In a typical example, primer sites were 18-21 bp in length, each unique barcode was 16 bp in length, and the UMI was 10 bp in length. Lengths for each of these regions can vary based on amplification conditions and level of desired multiplexing.

### Conjugation of Targeting Molecules to Nanoparticles

In an exemplary click chemistry reaction, nanoparticles were synthesized with a lipid containing a click chemistry handle such as an azide or methyl tetrazine. Liposomes were prepared using a standard thin film evaporation method, solvent precipitation method or microfluidic mixing method. For the thin film method, lipid components were combined with DSPE-PEG-tetrazine in a 2:1 chloroform/methanol mixture. The molar ratio of DSPE-PEG-tetrazine varied from 0.01% to 5% of the total lipid content. The organics were evaporated to form a thin film, which was dried overnight under reduced pressure. Aqueous buffer was added to hydrate the lipids and the film was sonicated in a bath sonicator at 60-70° C. for one hour. While still hot, the lipid solution was extruded through three successive syringe filters (0.45 μm, 0.2 μm, and finally 0.1 μm) to obtain liposomes. Unincorporated lipids were washed away using centrifugal filtration or tangential flow filtration (TFF) with a 100-300 kDa MWCO membrane. Tetrazine containing lipids can also be incorporated into liposomes using the post insertion method, where the DSPE-PEG-tetrazine is combined with an unfunctionalized nanoparticle following initial nanoparticle synthesis.

Specific targeting groups such as IgG, peptide, etc. were covalently modified using a complementary handle such as trans cyclooctene (TCO). For instance, an antibody can be nonspecifically modified at reactive amino acids or selectively modified at specific regions such as the C- or N-terminus, the hinge region, specific sugar residues, specific sequence sites, etc. In a typical nonspecific modification reaction, an IgG antibody at 0.1-1 mg/mL was combined with five equivalents of TCO-PEG4-NHS ester in the presence of 100 mM sodium bicarbonate buffer at room temperature. The reaction was allowed to proceed for one hour at room temperature, after which unreacted TCO was removed by centrifugation using 30 kDA MWCO filters. This process typically yielded 0.5-5 reactive TCO moieties per antibody. Nanoparticles bearing tetrazine moieties were reacted with complementary TCO-bearing antibodies to create specifically targeted nanoparticles. In a typical reaction, nanoparticles at 5×10¹² particles/mL were combined with TCO-antibody to achieve a final antibody concentration of 1 μM. The reaction was allowed to proceed overnight at room temperature. After the incubation, the unreacted antibody was removed via tangential flow filtration or centrifugation through a 300 MWCO filter.

### In Vitro Studies

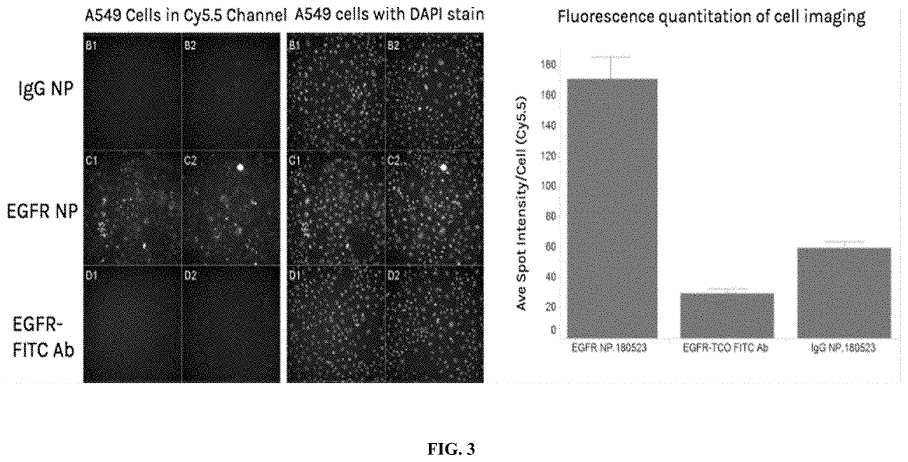

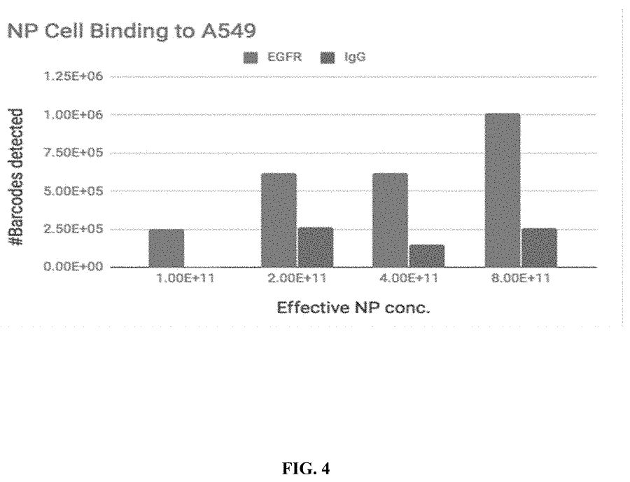

Specific targeting of antibody-functionalized nanoparticles to cells was demonstrated using anti-EGFR antibody labeled liposomes on A549 lung cancer cell lines. Antibody nanoparticles (with either anti-EGFR antibody or IgG isotype control) were synthesized as described above and tagged with Cy5.5 fluorescent dye. The nanoparticles were barcoded with ˜7 barcodes per particle as determined by qPCR and nanoparticle counting via Nanosight. Each particle was labeled with an average of 15-18 antibodies per particle. Cell binding was performed with EGFR positive A549 cells and imaged by Operetta (Perkin Elmer, Waltham, Mass.) in the Cy5.5 fluorescence channel. FIG. 3 (left panel and right panel) show cell imaging of targeted and barcoded nanoparticles in the presence of A549 cells and fluorescence quantitation of cell imaging, respectively. FIG. 4 shows specific binding of EGFR-NP with A549 cells as observed using qPCR. Briefly, A549 cells were incubated with nanoparticles at multiple concentrations for 2 hours at 37° C. Cells were washed with PBS, resuspended in PBS containing 0.25% Triton and immediately frozen. The cell mixture was thawed prior to qPCR and qPCR was performed with Kapa SYBR FAST qPCR Master Mix reagent.

### In Vivo Studies

The barcodes used were single-stranded DNA molecules of 72 nucleotides in length containing universal primer binding sites, a UMI sequence and a barcode sequence. Blood and tissues from mice injected with barcoded nanoparticles were recovered and weights were recorded. Barcodes were extracted using the Clarity OTX kit (Phenomenex, Torrance, Calif.) according to manufacturer's instructions. Briefly, Clarity OTX Loading/Lysis Buffer was added to blood (5:1 by volume) and incubated 15 minutes at room temperature. For tissues, Clarity OTX Loading/Lysis Buffer was diluted 1:1 with nuclease-free water and added to tissues for a 10 mg/mL final concentration. Tissue samples were homogenized using an OMNI THb tissue homogenizer. Tissue samples were incubated 5 min at room temperature to allow foam to settle prior to centrifugation at low speed to pellet tissue debris. Tissue lysate was transferred to a new tube and centrifuged at higher speed to remove any additional solids. Tissue lysate was collected and an internal barcode standard was added to all samples. Samples were loaded onto pre-equilibrated Clarity sorbent columns attached to a vacuum apparatus. A volume of equilibration buffer followed by three volumes of wash buffer were passed over the column prior to eluting with one column volume of elution buffer. Eluants were placed overnight into a centrivap at room temperature to dry samples. Samples are resuspended into Tris-EDTA buffer, pH 8.

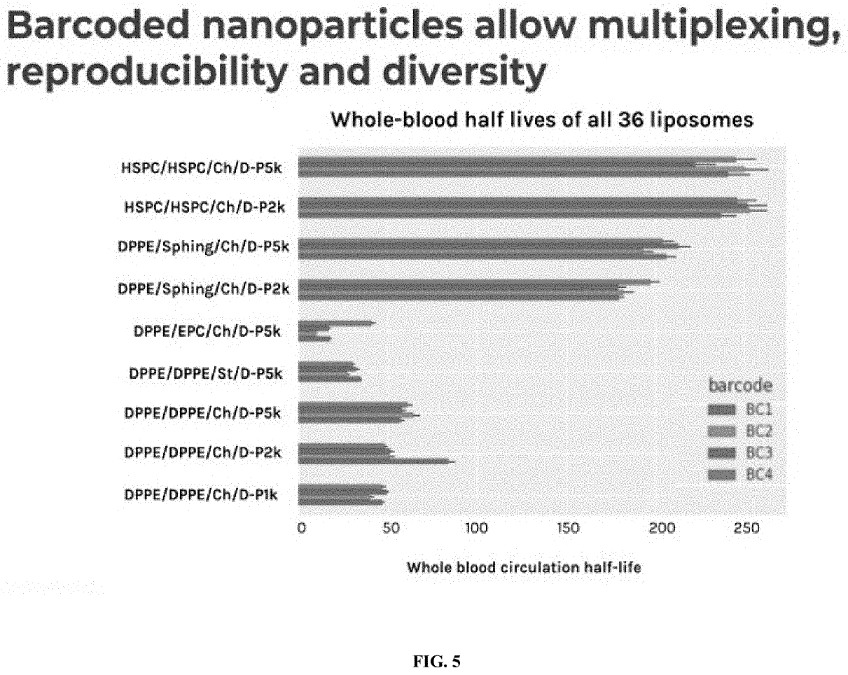

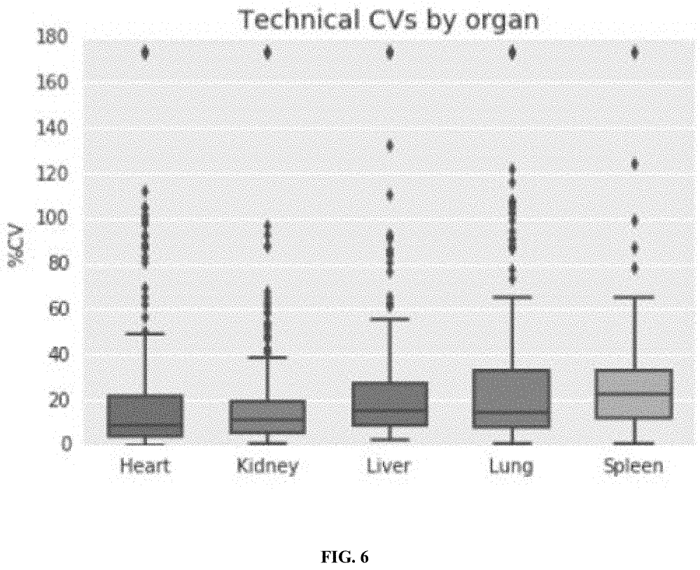

Resuspended samples were used as template for preparation of sequencing libraries. A small volume qPCR reaction with the Kapa SYBR FAST qPCR Master Mix reagent (ThermoFisher, Waltham, Mass.) and indexed primers designed against the universal primer binding sites in a BioRad CFX96 thermocycler (Hercules, Calif.) was set up. Larger volume PCR reactions set up as above were cycled using the Ct established at half-maximal intensity in the small volume reaction and resulting libraries were purified using AMPure XP Beads (Beckman Coulter Agencourt, Brea, Calif.), according to manufacturer's instructions, and eluted into elution buffer (Qiagen, Hilden, Germany). Fragment analysis using a Bioanalyzer and qPCR for concentration determination were performed. Libraries were pooled equally by concentration and prepared for sequencing on Illumina's MiSeq using a 31 cycle-single read with dual indexing protocol. Resulting reads were deconvoluted by indices and barcodes to link nanoparticle identity and sequence counts normalized to an internal standard. Analyses were performed for blood circulating half-life determinations, percent of injected dose in tissue and % CV across replicates. FIG. 5 shows the results of a representative determination of blood circulation half-life (minutes) of 36 liposomes. Nine different lipid formulations with four different barcodes for each lipid formulation were tested. FIG. 6 shows the results of a representative determination of % CV across technical replicates of tissue samples. Three technical sample-processing replicates were performed for each organ sample. Differences in median CV were observed between different organs.

What is claimed is:

1. A method for identifying one or more targeted nanoparticles suitable for delivery to one or more organs or tissues of a subject, comprising:
(a) administering to a subject a plurality of targeted nanoparticles, wherein the plurality includes subsets of nanoparticles, wherein each subset is conjugated to a unique non-lipid targeting molecule capable of binding a target antigen in an organ or tissue and wherein each subset comprises a barcode identifier sequence corresponding to the unique non-lipid targeting molecule;
(b) obtaining one or more cells from the one or more organs or tissues of the subject;
(c) amplifying one or more barcode identifier sequences in the one or more cells obtained in step (b); and
(d) identifying the one or more non-lipid targeting molecules associated with the barcode identifier sequence in the one or more cells from the one or more organs or tissues of the subject, thereby identifying one or more targeted nanoparticles suitable for delivery to one or more organs or tissues of the subject.

2. The method of claim 1, further comprising sequencing the amplified barcode identifier sequences from step (c).

3. The method of claim 1, wherein the plurality of targeted nanoparticles comprises between about two and about five hundred nanoparticles.

4. The method of claim 1, wherein the non-lipid targeting molecule is selected from the group consisting of an antibody, a protein, a peptide, an aptamer or a small molecule.

5. The method of claim 1, wherein the antigen is a protein, a lipid or a carbohydrate.

6. The method of claim 1, wherein each unique non-lipid targeting molecule binds a different target antigen.

7. The method of claim 1, wherein the plurality of nanoparticles comprises two or more nanoparticles, and wherein each unique non-lipid targeting molecule has a different binding affinity for the same target antigen.

8. The method of claim 1, wherein the plurality of nanoparticles comprises two or more subsets of nanoparticles, and wherein each subset of nanoparticles has a different size.

9. The method of claim 1, wherein the plurality of nanoparticles comprises two or more subsets of nanoparticles, and wherein each subset of nanoparticles has a different chemical composition.

10. The method of claim 1, wherein the plurality of nanoparticles comprises two or more subsets of nanoparticles, and each subset has a different number of unique non-lipid targeting molecule attached to each nanoparticle in the subset.

11. The method of claim 1, wherein the barcode identifier sequence further comprises a unique molecular identifier sequence.

12. The method of claim 11, wherein the method further comprises amplifying one or more unique molecular identifier sequences.

13. The method of claim 12, wherein the method further comprises sequencing one or more unique molecular identifier sequences.

14. The method of claim 1, further comprising quantifying the one or more barcode identifier sequences.

15. The method of claim 14, further comprising quantifying the one or more unique molecular identifier sequences.

16. The method of claim 1, wherein the barcode identifier sequence is between about five and about one hundred nucleotides in length.

17. The method of claim 1, wherein the unique molecular identifier sequence is between about five and about fifty nucleotides in length.

18. The method of claim 1, wherein the one or more cells are separated into individual compartments prior to amplification of the barcode identifier sequence(s).

19. The method of claim 1, wherein the one or more cells are lysed prior to amplification of the barcode identifier sequence.

20. The method of claim 2, wherein the amplification products are pooled prior to sequencing.

21. The method of claim 1, wherein the one or more cells are obtained from one or more tissues or organs selected from the group consisting of heart, liver, lung, muscle, blood, brain, spleen, skin, pancreas, mouth, esophagus, stomach, small intestine, large intestine, gallbladder and kidney.

22. The method of claim 21, wherein the one or more cells are cancer cells.
